(19) United States
(12) Patent Application Publication
(10) Pub. No.: US 2023/0326066 A1
(43) Pub. Date: Oct. 12, 2023

(54) CONTROL INTERFACE WITH OBJECT DISCRIMINATION

(71) Applicant: Linemaster Switch Corporation, Woodstock, CT (US)

(72) Inventors: William Chan, Brooklyn, CT (US); Sean Lewis, Woodstock, CT (US)

(21) Appl. No.: 17/714,636

(22) Filed: Apr. 6, 2022

(51) Int. Cl. G06T 7/70; G06T 7/20; B25J 19/02; B25J 9/16; G06F 3/02

(52) U.S. Cl. CPC G06T 7/70; G06T 7/20; B25J 19/023; B25J 9/1697; G06F 3/02

(57) ABSTRACT

A control interface includes a sensor having a sensing field projecting adjacent to one or more input devices such as switches actuated by an operator. The control interface detects the profile, position, and/or motion of objects in the sensing field and compares these to stored values of profile, position, and/or motion representing a valid operator attempt to actuate the input device. Upon detection of a valid attempt to actuate the input device, the control interface generates a control signal that may be communicated to an operator or to connected equipment. The control signal is generated before operator makes contact with the input device. The control signal may be used to provide a pre-activation alert to the operator with identifying the input device about to be actuated. Alternatively, the control interface may disable input devices until a valid attempt to actuate the input device is detected, and the control signal is used to enable the input device.

### BACKGROUND

The present disclosure relates generally to control interfaces for industrial and medial equipment, including controls actuated by hands and/or feet of an operator, and more particularly, to a control interface that detects the form and movement of objects to prevent unintended actuation.

Safety is a critical issue in the design of control interfaces for industrial, transportation, and medical equipment. Many types of equipment pose significant hazards to equipment operators, medical patients, and the public. For example, it is known to design control interfaces for industrial equipment that require both hands on the control interface to cycle equipment such as a press, so the press cannot be cycled unless the operator's hands are in a safe position. Control interfaces for industrial equipment may include controls operable by a hand or a foot and may be provided with mechanical cover or hood that prevents unintended actuation of the control interface.

Surgical equipment may present unique safety issues. It is common for a medical treatment or surgical suite to include a range of equipment that require the technician or doctor to use a foot to actuate a control device while the technician or surgeon uses both their hands. The foot operated control device may actuate a laser for cutting, an electro cauterizing tool for cutting or cauterizing flesh, drills, saws or other instruments. These surgical instruments may be individual devices or part of a tower or rack that includes several different devices. To cut down on the number of cables, it is known to connect a multifunction footswitch to one or more pieces of equipment using a wireless interface. It is also known to use the same wireless footswitch to control several pieces of equipment. It is apparent that the footswitch is in a location where it is not visible by the technician or surgeon, whose attention is rightly focused on the patient and the procedure. This situation presents several critical safety issues. First, it is necessary for the user to know which device is connected to the footswitch. Further, the operator needs to know which function of the connected device is active, such that activation of the footswitch will have the intended result. In many cases, the footswitch has several pedals and/or switches at different locations on a base. These different switches and pedals activate different functions, so it is important for the operator to know the location of the foot relative to the different pedals and switches to ensure activation of the intended switch or pedal. With existing footswitches, this may require taking the operator's eyes off the procedure to confirm accurate foot placement.

It will be apparent that accurate control of these surgical instruments is critical to patient safety and successful patient outcomes.

There is a need for an improved control interface that provides reliable feedback to an operator of the equipment regarding the equipment connected to the control interface and which function of the connected equipment will be activated by a control input.

There is a need for an improved control interface that can sense object attributes and movement to prevent accidental or unintended actuation of connected equipment.

### SUMMARY OF THE INVENTION

According to aspects of the disclosure, a control interface includes a sensor having a sensing field projecting adjacent to one or more input devices such as switches actuated by an operator. The control interface detects the profile, position, and/or motion of objects in the sensing field and compares these to stored values of profile, position, and/or motion corresponding to a valid operator attempt to actuate the input device. Upon detection of a valid attempt to actuate the input device, the control interface generates a control signal that is either used in the control interface itself or communicated via a wired or wireless communications link to an operator or to connected equipment. The control signal is generated before operator makes contact with the input device. The control signal may be used to provide a pre-activation alert to the operator with information about the input device about to be actuated. Alternatively, the control interface may disable or “lockout” input devices until a valid attempt to actuate the input device is detected, and the control signal can be used to enable the input device.

The disclosed control interface can distinguish between a valid attempt to actuate an input device such as the hand or foot of an operator in the sensing field and other objects in the sensing field. This object discrimination feature can be used to enhance the safety of equipment connected to the control interface by providing the operator with information about an action about to be taken before the action is commenced. The information provided to the operator can include the equipment and/or function connected to the input device for which a valid attempt has been detected. This allows the operator to confirm that the connected equipment and/or function is that which the operator intends to actuate. One example is a medical instrument that can cut or cauterize. It is obviously important that the operator ensure the function about to be actuated is the intended function.

The control interface may have one or more sensors and one or more input devices. One sensor having a single sensing field may be arranged to extend adjacent more than one input device and data corresponding to the profile, position and/or motion of objects in the sensing field may be collected and compared to stored values for profile, position and/or motion corresponding to a valid attempt to actuate each of the input devices. When the sensed profile, position and/or motion of an object in the sensing field matches the profile, position and/or motion of a valid attempt to actuate one of the input devices, a control signal is generated. The control signal may be used to provide an operator with information identifying the input device for which a valid attempt has been detected and this information may be communicated to the operator from the control interface itself or through equipment such as s surgical console connected to the control interface.

In some embodiments, the control interface may be configured to disable or lockout an input device until a valid attempt to actuate the input device is detected. Disabling input devices until a valid attempt to actuate the input device is detected will prevent some accidental actuations of the input device such as by an object falling on a footswitch. In some embodiments, the control interface may be configured to delay enabling an input device to provide time for a pre-activation warning to be delivered to the operator. In other embodiments, the control interface may be configured to require acknowledgment of the pre-activation warning before the input device is enabled.

The disclosed control interface includes a processor with memory that may take the form of a microcontroller. The processor is programmed to execute an algorithm that compares one or more attributes of an object in the sensing field to stored values of attributes of an object in the sensing field corresponding to a valid attempt to actuate an input device on the control interface. According to aspects of the disclosure, the stored values may be collected by configuring the control interface as desired, arranging the control interface in a position corresponding to its use environment and then placing objects corresponding to valid attempts to actuate the input device in the sensing field. For a control device to be used as a footswitch, the control interface would be placed on the floor and exposed to various shoe-clad and/or bare feet to gather data that can be used to generate the stored values. For a control device to be used as a hand-operated switch, the control interface would be mounted in its intended use position and exposed to a variety of human hands or human hands in gloves to gather data that can be used to generate the stored values. The stored values will typically include a range of profiles, positions and/or motions to accommodate differences among operators.

The disclosed control interface is programmable and can be configured to suit a particular application or use environment. Object attributes can be changed as needed. In some embodiments, the motion of an object may not be important and the values for this object attribute may be ignored when making the comparison. In other embodiments the profile of the object include any or most profiles so that almost any shape object in the correct position and having a correct motion will result in a recognized attempt to actuate the input device. In some embodiments, the algorithm executed by the processor requires comparison of the profile, position and motion to stored values for profile, position and motion corresponding to a valid attempt to actuate an input device on the console and generate a control signal only when all of the object attributes of profile, position and motion fall within the stored values. In other embodiments, the algorithm may generate the control signal when the attributes of the object fall within the stored values for only one of the profile, position or motion of an object corresponding to a valid operator attempt to actuate an input device. In still further embodiments, the algorithm will generate the control signal when at least two of the profile, position or motion of an object fall within the stored values for profile, position and motion of an object corresponding to a valid attempt to actuate a control device.

### BRIEF DESCRIPTION OF THE DRAWINGS

FIG. 1 is a schematic representation of one embodiment of a control interface according to aspects of the disclosure;

FIG. 2 is a right side elevation view of an embodiment of a control interface according to aspects of the disclosure in functional conjunction with the foot of an operator of the control interface;

FIG. 3 is a top perspective view of the control interface of FIG. 2 showing sensing fields and zones of object detection above the control interface according to aspects of the disclosure;

FIG. 4 is a top plan view of the control interface of FIGS. 2 and 3;

FIG. 5 is a left side elevation view of the control interface of FIG. 2;

FIG. 6 is a front elevation view of the control interface of FIGS. 2-5 with the operator's foot removed and showing sensing fields and zones of object detection above the control interface according to aspects of the disclosure;

FIG. 7 is a flow chart illustrating a representative program for detecting an operator input to the disclosed control interface and generating a pre-activation warning to the operator according to aspects of the disclosure;

FIG. 8 is a flow chart illustrating an alternative program for detecting an operator input to the disclosed control interface and enabling an input device according to aspects of the disclosure;

FIG. 9 is a flow chart of a representative algorithm for use in an alternative embodiment of the disclosed control interface;

FIG. 10 is a flow chart illustrating a first variation of the algorithm of FIG. 9;

FIG. 11 is a flow chart illustrating a second variation of the algorithm of FIG. 9;

FIG. 12 is a flow chart illustrating a third variation of the algorithm of FIG. 9;

FIG. 13 is a flow chart illustrating a representative algorithm for use in an alternative embodiment of the disclosed control interface;

FIG. 14 is a flow chart illustrating a first variation of the algorithm of FIG. 13;

FIG. 15 is a flow chart illustrating a second variation of the algorithm of FIG. 13;

FIG. 16 is a flow chart illustrating a third variation of the algorithm of FIG. 13; and

FIG. 17 illustrates a representative X-Y-Z three-dimensional coordinate system and shows a human hand in a three dimensional coordinate system.

### DETAILED DESCRIPTION

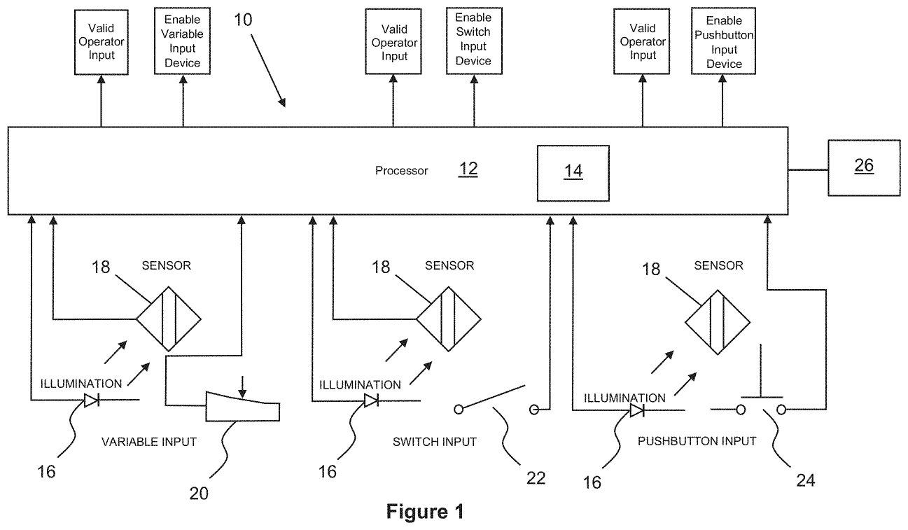

A control interface 10 according to aspects of the disclosure will now be described with reference to the figures. FIG. 1 is a schematic diagram illustrating the functional units and connections in an embodiment of a control interface 10. A processor 12 with memory 14 is connected to at least one LED 16 (or illuminator), at least one sensor 18, input devices such as switches 20, 22, 24 and a communications link 26. The communications link 26 may be wired or wireless, and in either form allows the control interface 10 to communicate with connected equipment (not shown). In some embodiments, the control interface 10 may be constructed to communicate directly with the operator by audible, visual, haptic or other means. The LED 16 generates light under control of the processor 12 to illuminate a three-dimensional space adjacent the control interface 10. The LED 16 and sensor 18 are matched, with one example being a sensor that detects infrared (IR) light and LEDs that generate IR light. The LED(s) 16 are oriented to project light away from the control interface 10 so the light reflects from objects as the objects approach the control interface 10. The control interface 10 may be a footswitch arranged on a floor or other support surface and include one or more input devices such as switches 20, 22, 24 to be actuated by a human foot. Alternatively, the control interface 10 may support a hand operated input device, in which case the control interface 10 is supported in a position where it is accessible by a human hand. The orientation of the control interface 10 may be horizontal, vertical or at any desired orientation selected to facilitate access by an operator. In any orientation, the LED(s) 16 illuminate an area projecting away from the input devices 20, 22, 24, where the illuminated area must be penetrated by a hand or foot as it approaches the control interface 10 to actuate an input device 20, 22, 24 supported on the control interface 10.

FIG. 1 illustrates a control interface 10 with three sensors 18 paired with three LEDs 16. It is not necessary that each sensor 18 have its own LED 16, but in this disclosed sensor arrangement at least one LED 16 or source of illumination is necessary. Some embodiments may illuminate a sensing field for multiple sensors with a single LED 16 of sufficient intensity and emission pattern. Alternatively, embodiments may employ a single sensor 18 detecting a sensing field illuminated by multiple LEDs 16. FIG. 1 illustrates different input devices 20, 22, 24 that may be provided on a control interface 10. Input device 20 is a variable input such as a potentiometer that can be connected to a foot pedal or hand operated lever actuated by an operator to control speed or power of a connected device. Input device 22 is a two-position switch movable between a first position and a second position where the first position may correspond to an open position and the second position may correspond to a closed position or the first position may correspond to a first connection and the second position corresponds to a second connection. A two-position switch such as switch 22 maybe used to alternatively connect devices or functions to the control interface 10. Switch 22 may have more than two positions and may be configured to alternatively connect a plurality of devices or functions of a device to the control interface 10. A multi position switch may be used to alternatively connect different equipment or functions of connected equipment to other input devices on the control interface 10. Input device 24 may be a push button switch such as a momentary switch that is closed when the switch is pressed by a hand or foot and then open when the switch is released. A momentary input device such as 24 may provide an input to the processor 12 where the processor is programmed to carry out a function or sequence in response to the input. Input devices compatible with the disclosed control interface 10 are not limited to the exemplary input devices 20, 22, 24 shown.

One embodiment of a disclosed control interface 10 will be described by reference to light generating LEDs and light detecting sensors but the disclosed control interface 10 is not limited to this emitter/sensor combination. The devices and methods described in this application may be adapted to employ any sensing methods that will provide information about objects in the sensing field with sufficient speed and detail to allow discrimination of objects in the sensing field or fields. Alternative sensing formats may include ultrasonic emitter/sensor, electromagnetic emitter/sensor or an optical, microwave, or acoustic sensor, and often, a transmitter for illumination. Sensors may be active and include an emitter generating an emission that “illuminates” an object and the reflected emission is detected by the sensor. A passive system may rely on ambient conditions or emission such as heat from an object. A sensor may be a single receptor or an array of receptors connected together. An array of receptors can provide more detailed information about an object than a single receptor. For example a 6×10 array of optical sensors can form a pattern representative of an object in the sensing field. An array of sensors can also provide information about the position of an object and/or the motion of an object that may not be possible with a single optical sensor.

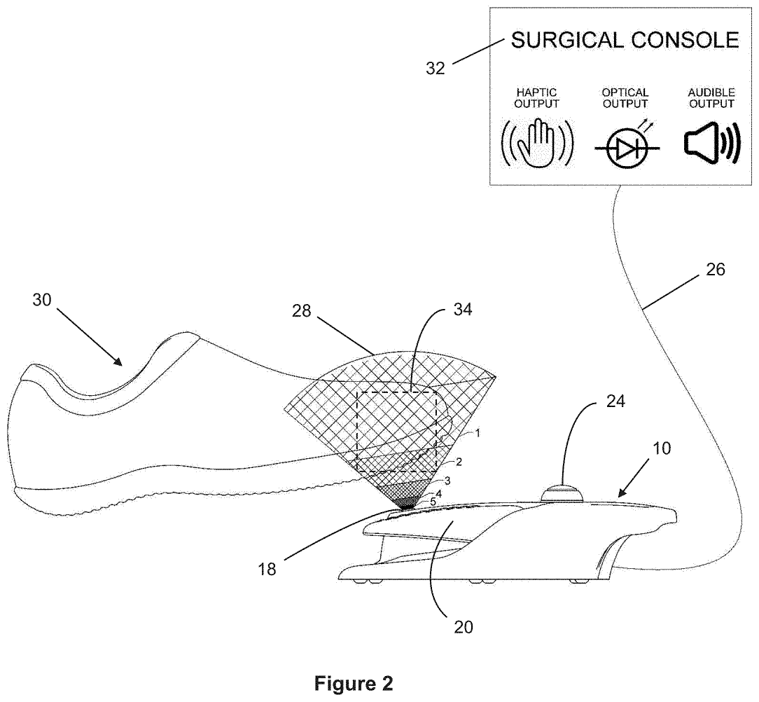

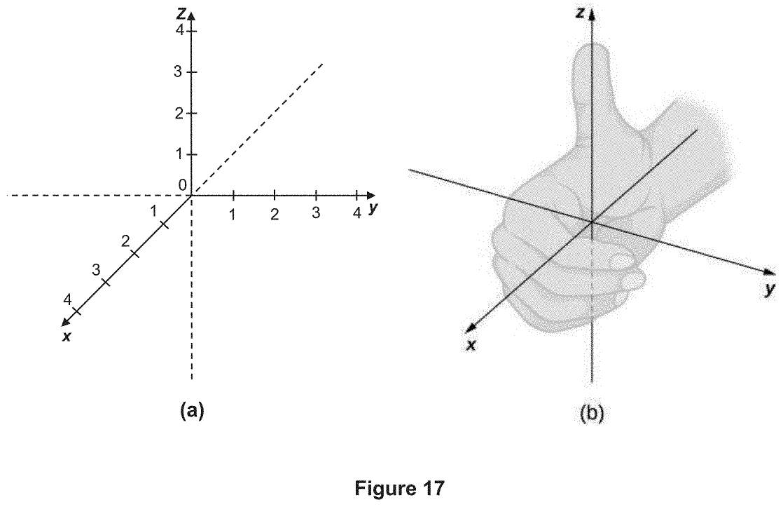

As shown in FIGS. 2-6, each sensing field 28 extends in three dimensions adjacent the control interface 10 and an object 30 (such as the hand or foot of an operator) must penetrate the sensing field to contact the control devices 20, 22, 24 supported on the control interface 10. FIG. 17 illustrates a three-dimensional coordinate system and a human hand within the coordinate system. As an object such as a human hand or foot penetrates a sensing field, the sensor(s) 18 are able to detect attributes of the object such as the shape or profile of the object, the position of the object relative to the sensor 18 and movement of the object relative to the sensor 18. According to aspects of the disclosure, these attributes of the object will be used to distinguish a valid attempt by an operator to actuate one of the input devices 20, 22, 24 from other objects that may penetrate the sensing field 28. An example of an object that might penetrate a sensing field of the control interface is a tool dropped by an operator of equipment connected to the control interface 10. The tool will accelerate as it falls and may contact one or more of the input devices 20, 22, 24. The tool will have attributes of shape or profile, or motion that will be distinct from attributes of an operator's hand or foot. According to aspects of the disclosure, the processor 12 is programmed to compare one or more attributes of objects in the sensing field 28 to one or more set of attributes stored in memory 14 that are known to represent a valid attempt to actuate one of the input devices 20, 22, 24. The comparison allows the disclosed control interface 10 to distinguish between the falling object and the operator's hand or foot and take one or more actions based upon the result of the comparison.

With reference to FIG. 17, a hand or foot of an operator is a three-dimensional object 30 that will produce a recognizable set of attributes when present in one or more of the sensing fields 28. The attributes of the object include its basic shape, which may be referred to in this disclosure as the “profile” of the object 30. “Profile” as used in this disclosure refers to the shape of the object in three dimensions and encompasses both the shape of the object in an X-Y plane as well as the depth of the object 30 with respect to the Z direction perpendicular to the X-Y plane. The sensor(s) 18 will detect the profile, position and motion of the object 30 in the sensing field and the processor will use one or more of these attributes in a comparison to stored values corresponding to a valid operator attempt to actuate a control device 20, 22, 24 on the control interface 10. The processor 12 is programmed to use the results of the comparison to take pre-determined actions.

With reference to FIGS. 2-6, one or more sensors 18 are arranged on the control interface 10 to detect light reflecting off objects 30 as they penetrate the space near the input devices 20, 22, 24. Each sensor 18 may include an array of sensing units or receptors that are connected to provide information in the form of adjacent pixels. As an object moves through the sensing field, sensing units will be illuminated in a pattern that allows the position or motion of an object to be detected. Each sensing unit may be capable of detecting intensity of emission reflected from an object and an array of sensors allows the proximity and profile or shape of the object to be detected by placing the detected intensities next to each other to form a pixelated image of the object. Together, the LED(s) 16 (or other source of illumination) and sensor(s) 18 function as a three-dimensional sensing field 28 having a depth projecting away from the input devices 20, 22, 24 (the Z direction), and a width and height in an X-Y plane perpendicular to the depth of the sensing field 28. FIG. 2 illustrates a sensing field 28 extending in the Z-direction away from the control interface 10. The sensor 18 is able to detect the position of the object 30 relative to the input devices 20, 24 and distinguish between an attempt to actuate input device 20 from an attempt to actuate input device 24. The ability to distinguish between attempts to actuate different input devices 20, 24 on a control interface can be an important safety feature, as will be described in greater detail below. Objects 30 in the sensing field reflect light that is detected by the sensor 30. Objects of different types in the sensing field 28 have different attributes that produce light reflection patterns that are detected by the sensor(s) 18. Attributes of objects 30 in the sensing field include, but are not limited to profile, position and motion or movement including direction and velocity or speed of the object 30. Attributes of objects like a hand or a foot in the sensing field 28 have a range of values that can be measured to determine properties of a human hand or foot approaching the input devices 20, 22, 24. Measured attributes of hands and feet can be used to generate a library of stored criteria representing what are considered “valid” attempts to actuate an input device 20, 22, 24. Objects 30 in the sensing field 28 that have the attributes of a valid attempt to actuate an input device can be distinguished from objects with attributes falling outside values corresponding to a valid attempt to actuate the input devices 20, 22, 24. For example, a hand or foot will have attributes that are distinct from an object dropped on a footswitch or leaning against a hand switch.

According to aspects of the disclosure, the processor 12 is connected to the LED(s) 16 to control the pattern and intensity of light generated by the LED(s) 16 and connected to the sensor(s) 18 to receive data corresponding to attributes of objects 30 in the sensing field 28. The processor 12 also includes or is connected to memory 14 in which the library of values for one or more attributes corresponding to valid attempts to actuate a control device 20, 22, 24 are stored. The processor 12 is programmed to run an algorithm which compares one or more of the attributes of objects 30 in the sensing field 18 with the stored values of one or more attributes corresponding to a valid attempt to actuate an input device 20, 22, 24. When one or more of the attributes of an object 30 in the sensing field 28 fall within the stored values for a valid attempt to actuate an input device 20, 22, 24, the processor 12 is programmed to generate a control signal. According to aspects of the disclosure, the control signal may be used to provide a “pre-activation” warning to an operator of equipment connected to the control interface 10. The pre-activation warning may be emitted directly from the control interface 10 in one or more forms sensible by a human operator including vibration, audible tone or sound, and/or visible light. The pre-activation warning alerts the operator that they are about to actuate a control device 20, 22, 24. The control output from the control interface 10 may be communicated to equipment connected to the control interface 10 via a wired or wireless communications link 26, so that the pre-activation warning is generated by the connected equipment such as a surgical console 32, rather than directly from the control interface 10 itself.

One example of a use for the disclosed control interface 10 is as a footswitch as shown in FIGS. 2-6 connected to a surgical console 32 in an operating room or surgical suite. The connected surgical equipment may be a single piece of equipment with multiple functions, such as equipment that can cut or cauterize flesh depending on the active function. Alternatively, there may be several different pieces of surgical equipment or tools that can be controlled using the control interface 10. In either scenario, it is critical that the operator using the control interface 10 be aware of which function or tool is connected to the control interface 10 so that actuation of a control device 20, 22, 24 has the intended effect. For example, the operator of a tool that can cut or cauterize needs to know which function is “active” so that actuation of the control device 20, 22, 24 carries out the intended function. In this use environment, the disclosed control interface 10 can be used to improve safety and patient outcomes by providing a pre-activation warning to the operator that they are about to actuate a control device 20, 22, 24. The pre-activation warning may include information regarding which function and/or tool is connected to be controlled by the control device 20, 22, 24 about to be actuated. Providing this information to the operator allows the operator to confirm the correct function or equipment is selected before the control device 20, 22, 24 is contacted by the foot or hand, by which time it may be too late to prevent an unintended result.

FIGS. 2-6 illustrate a control interface in the form of a footswitch supporting three input devices including two variable input devices 20 and one momentary push button switch 24 located in the center of the control interface 10. FIG. 2 illustrates a control interface 10 with a single sensor 18 arranged to detect objects in a sensing field adjacent the control interface 10. The sensing field 28 is represented in the form or a conical portion of a sphere projecting above the control interface 10, but the sensing field 28 is not limited to this shape and may not have well-defined margins as shown in the Figures. It will be apparent to those skilled in the art that the sensing field 28 will need to extend over all the input devices 20, 24 to enable detection of attempts to actuate each of the input devices 20, 24. This can be accomplished by selection and positioning of the sensor 18 as well as selection and positioning of one or more LEDs 16 to illuminate objects in the sensing field 28. The sensing field 28 in FIG. 2 is shown in a vertical plane extending in the Z direction projecting away from the control interface 10. Within the sensing field 28 a square represents a range of positions 34 of an object 30 in the vertical plane corresponding to a valid attempt to actuate one of the variable pedal switches 20. It will be understood that the square shape used to represent a range of positions 34 in the vertical plane is merely an illustration and a projection of the actual range of positions corresponding to a valid attempt to actuate switch 20 may be asymmetrical or any shape when projected within the vertical plane. The vertical plane of FIG. 2 is taken along a direction parallel with one of the X or Y axes of a three-dimensional coordinate system as illustrated in FIG. 17. Whatever the shape of the projection of positions in the vertical plane corresponding to a valid actuation attempt, the range of positions is stored in memory 14 and used in the comparison algorithm executed by the processor 12 according to aspects of the disclosure.

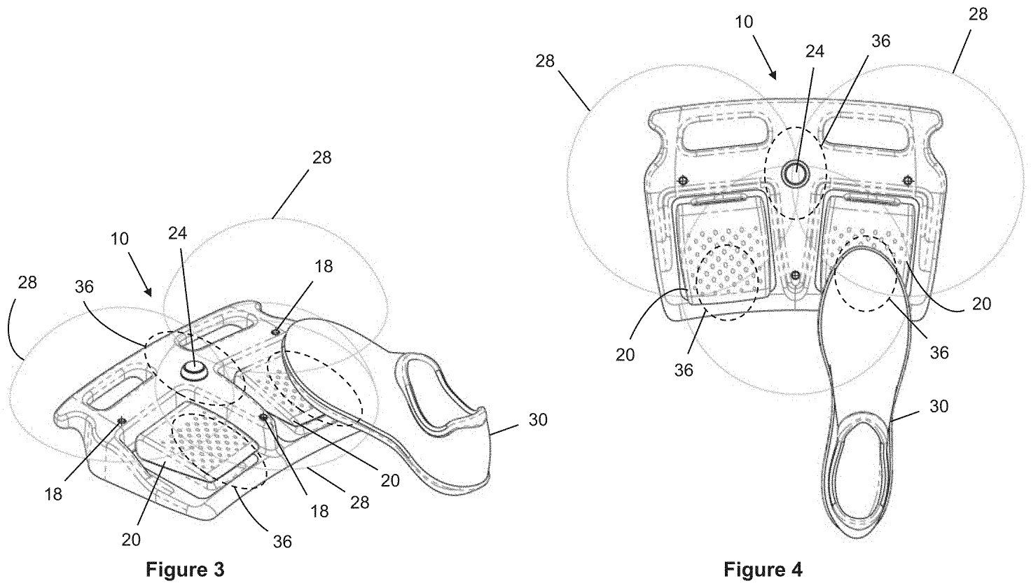

FIGS. 3-6 illustrate an embodiment of a control interface 10 with three sensors 18, each having a sensing field 28 extending above the control interface 10. Again, each sensing field 28 is represented as a conical portion of a sphere, but this is only a convenient way of visualizing the sensing fields 28. As shown in FIGS. 2-6 the sensing fields 28 of the three sensors 18 overlap in planes corresponding to the X-Y-Z directions of a three-dimensional coordinate system. Ovals in FIGS. 3 and 4 are used to represent a range of object positions 36 in the X-Y plane corresponding to a valid attempt to actuate each of the three input devices 20, 24, 20, respectively. The ovals in FIG. 4 are taken in a plane containing the X-Y axes of the coordinate system, while the ovals in FIG. 4 are taken at an angle to the X-Y plane. The ovals are a convenient visualization of the range of object positions 36 in the X-Y plane corresponding to valid attempt to actuate each of the input devices 20, 24, 20. The actual shape of the range of valid positions projected in the X-Y plane may be any shape and is not limited to the representative ovals used in FIGS. 3 and 4. It is important to note that the range of object positions 36 corresponding to a valid attempt to actuate each of the input devices 20, 24, 20 do not overlap, so the illustrated embodiment of the control interface 10 can distinguish between an attempt to actuate one of the switches from an attempt to actuate the other switches. This is an important safety feature of the disclosed control interface 10 and allows the control interface 10 to generate a control signal corresponding to a valid attempt to actuate each of the three input devices 20, 24, 20 on the control interface 10. Each of the input devices 20, 24, 20 may be used for a different function or purpose with regard to the equipment connected to the control interface 10 and the control interface 10 can use the different control signals to alert the operator of the equipment which of the three input devices 20, 24, 20 they are about to actuate. According to aspects of the disclosure the actuation attempt and alert can be delivered to the operator before the input device is actually contacted, potentially preventing an unintended action by the operator.

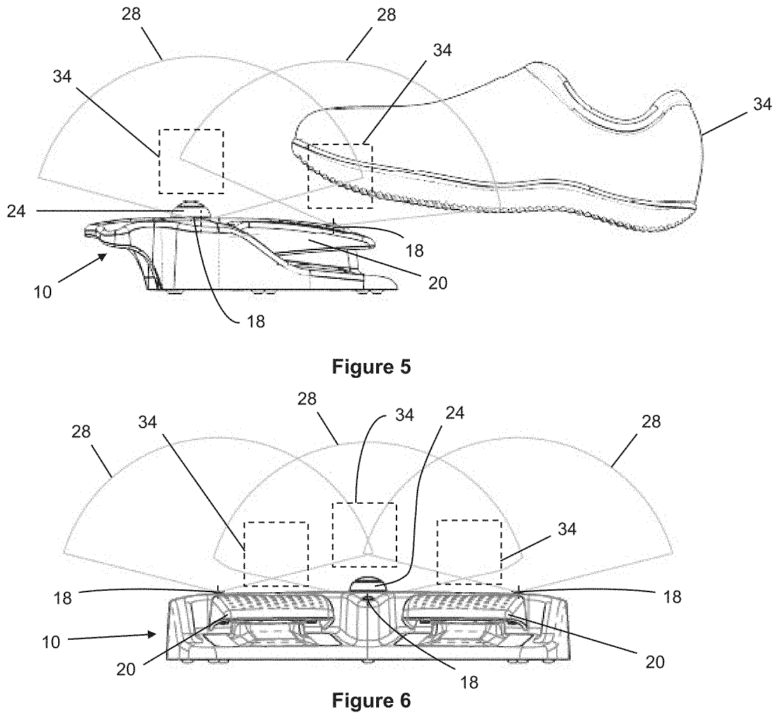

FIGS. 5 and 6 are left side and front elevation views of the control interface of FIGS. 3 and 4. In FIGS. 5 and 6, squares are used to represent a range of positions 34 in planes parallel with the Z direction of the coordinate system corresponding to a valid attempt to actuate either one of the variable input devices 20 or the momentary input device 24. The plane of FIG. 5 is perpendicular to the vertical plane of FIG. 6. The square shape is merely a convenient representation of the range of valid positions 34 corresponding to a valid attempt to actuate each of the three input devices 20, 24, 20. It is understood that the actual shape of the range of valid positions 34 projected in the planes of FIGS. 5 and 6 is not limited to the square shape shown and importantly may be a different shape in each plane. The range of valid positions 34 for each of the three input devices do not overlap, so the control interface 10 is able to distinguish an attempt to actuate one of the input devices from an attempt to actuate the other input devices.

With reference to FIGS. 3-6, the sensing fields 28 of the three sensors 18 overlap in each of the directions X-Y-Z corresponding to the coordinate system, with the result that the stored values of valid positions 34, 36 may include values generated by more than one sensor 18. Position values corresponding to valid attempts corresponding to positions with respect to more than one sensor 18 can be stored in memory 14 and compared to data collected from more than one sensor 18 to detect valid actuation attempts according to aspects of the disclosure. It will be observed that a centrally located sensor 18 can have a sensing field 28 that extends over all of the input devices 20, 24, 20 and could be used to detect objects with respect to all three input devices 20, 24, 20 in a control interface 10 that employs only a single sensor 18. Multiple sensors 18 may be employed to improve the accuracy of object sensing with respect to the control interface 10. While the sensing fields 28 for a plurality of sensors 18 supported on the control interface 10 may overlap, the range of object positions corresponding to a valid attempt to actuate each of the three input devices 20, 24, 20 do not overlap, ensuring that the control interface will not confuse an attempt to actuate one of the input devices 20, 24, 20 with an attempt to actuate one of the other input devices 20, 24, 20.

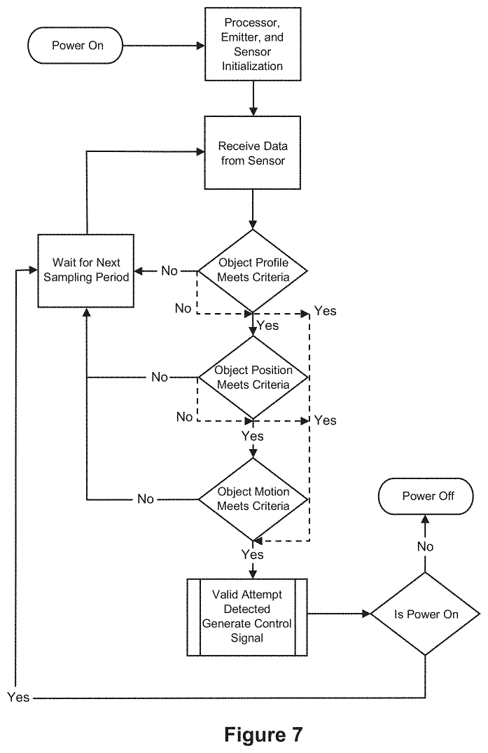

According to aspects of the disclosure, in one embodiment of a control interface, the control signal generated upon detection of a valid attempt to actuate an input device is used to generate a pre-activation warning to the operator of equipment connected to the control interface 10. When the profile, position and/or motion attributes of an object in the sensing field(s) meet the criteria of a valid attempt to actuate an input device, a pre-activation warning can be provided to the operator before the input device is contacted to initiate a function of the connected equipment. The timing of the pre-activation warning allows the operator an opportunity to confirm the intended function and or equipment and avoid an unintended and possibly harmful action. In this embodiment, the input devices 20, 22, 24 are enabled and contact with the input devices 20, 22, 24 will initiate a function of the connected equipment. FIG. 7 is a flow chart illustrating representative steps in an algorithm executed by the processor 12 to compare the attributes of an object in the sensing field(s) to stored attributes corresponding to a valid attempt to actuate one of the input devices. It will be understood that if the control interface 10 includes more than one input device 20, 22, 24, then the stored criteria will include attributes of a valid attempt to actuate each of the input devices 20, 22, 24. When the attributes of an object in the sensing field(s) meets the profile, position and/or motion criteria for a valid attempt to actuate one of the input devices, the control interface 10 generates a control signal corresponding to the specific input device. A pre-activation warning to the operator may include information about which input device is about to be actuated, allowing the operator to confirm what is about to happen before the input device is contacted. The solid lines in FIG. 7 illustrate a comparison algorithm where all of the profile, position, and motion attributes of an object in the sensing field are required to match the stored values of profile, position and motion corresponding to a valid attempt to actuate one of the input devices 20, 22, 24 before the control signal is generated. This algorithm may prove too restrictive for some applications, so FIG. 7 shows an alternative algorithm represented by the dashed lines. According to the dashed lines in FIG. 7, if any one of the profile, position, or motion attributes of an object in the sensing field(s) match the stored values of profile, position, or motion corresponding to a valid attempt to actuate one of the input devices 20, 22, 24, then the control signal is generated. A further alternative algorithm may generate the control signal only if at least two of the profile, position or motion attributes of an object in the sensing field(s) matches the stored values of profile, position or motion corresponding to a valid attempt to actuate one of the input devices 20, 22, 24. The control interface 10 is programmable and the algorithm can be adjusted to suit different installed orientations or uses by making the comparison algorithm more or less restrictive.

One use for the disclosed control interface 10 is in an industrial environment where the control interface 10 is connected to a piece of industrial equipment such as a press or CNC machine. The pre-activation warning can alert the equipment operator that they are about to contact a control device that will initiate a cycle or function of the connected equipment. For example, if the equipment has different cycles “A” or “B”, the pre-activation warning may include information about the cycle that is about to be initiated to allow the operator to confirm it is the correct cycle. Initiating the incorrect cycle may produce an unsafe condition or destroy valuable raw materials or parts.

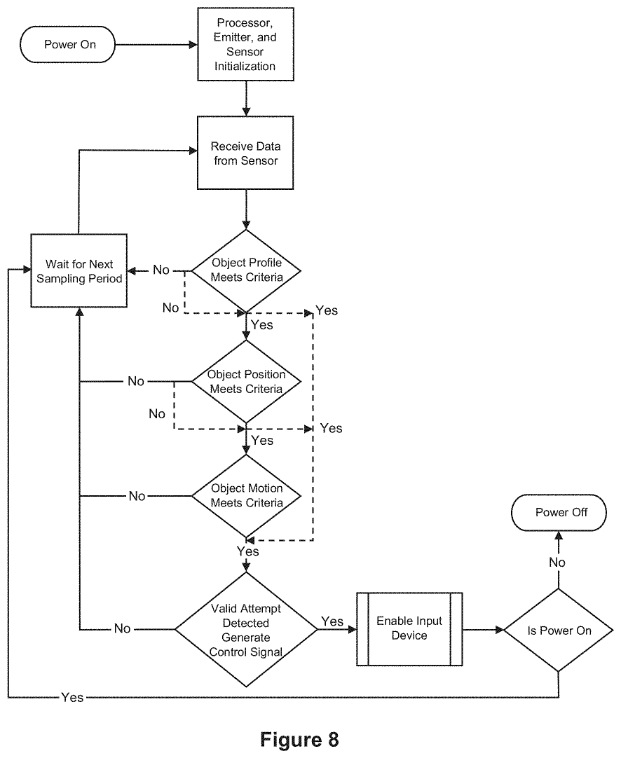

FIG. 8 is a flow chart illustrating representative steps in an algorithm executed by the processor 12 to compare the attributes of an object in the sensing field(s) to stored attributes corresponding to a valid attempt to actuate one of the input devices. In the control interface of FIG. 8, the input devices 20, 22, 24 are disabled until a valid attempt to actuate one of the input devices is detected. This “lockout” feature prevents accidental actuation of an input device 20, 22, 24 by an object dropped or accidentally coming into contact with an input device 20, 22, 24. The algorithm of FIG. 8 shown in solid lines compares the attributes of an object in the sensing field(s) to stored attributes corresponding to a valid attempt to actuate one of the input devices. It will be understood that if the control interface 10 includes more than one input device 20, 22, 24, then the stored criteria will include attributes of a valid attempt to actuate each of the input devices 20, 22, 24. When the attributes of an object in the sensing field(s) meet the profile, position and motion criteria for a valid attempt to actuate one of the input devices, the control interface 10 generates a control signal corresponding to the specific input device. In this embodiment of a control interface 10, the processor is programmed to use the control signal to enable the input device for which a valid actuation attempt has been detected. Safety is enhanced when the input devices are disabled until an object meeting the criteria for a valid actuation attempt is detected. FIG. 8 illustrates an algorithm in dashed lines where the control signal is generated if any one of the profile, position, or motion attributes of an object in the sensing field(s) results in generation of the control signal corresponding to a valid attempt to actuate one of the input devices 20, 22, 24. The algorithm can also be configured to generate the control signal when two of the three attributes of profile, position or motion match the stored values for profile, position or motion corresponding to a valid attempt to actuate one of the input devices 20, 22, 24. The algorithm can be made more or less restrictive depending upon the end use of the control interface 10. The algorithm can also be adjusted by making the range of stored values of profile, position or motion corresponding to a valid attempt more, or less restrictive. In one possible scenario, the algorithm still compares all three attributes of profile, position and motion, but one or more of the range of stored values is made less restrictive, making the algorithm less restrictive. In an alternative scenario, the stored values corresponding to one or more of the profile, position or motion are made more restrictive, making the algorithm more restrictive.

When the control interface 10 includes more than one input device 20, 22, 24, the stored values corresponding to a valid attempt to actuate an input device will include criteria for valid attempts corresponding to each of the input devices 20, 22, 24 as illustrated in FIGS. 3-6. The input devices 20, 22, 24 will necessarily occupy different physical positions on the control interface 10, so objects 30 in the sensing field(s) 28 approaching one of the input devices 20, 22, 24 will have different attributes of direction of movement and position relative to objects 30 in the sensing field(s) 28 approaching other of the control devices 20, 22, 24. According to aspects of the disclosure, the criteria stored in memory will include values corresponding to attributes of an object 30 representative of a valid attempt to actuate each of the control devices 20, 22, 24. The attributes of a valid attempt for one control device will not overlap with the values of a valid attempt for any of the other control devices. The algorithm includes comparisons of the attributes of objects 30 in the sensing field(s) to the stored values corresponding to valid attempts for each of control devices 20, 22, 24 and include a step of generating a control signal when a valid attempt for one of the input devices 20, 22, 24 is detected. The control signal may be used to provide a pre-activation warning to the operator including information about which input device is about to be actuated. For example, if a valid attempt to actuate the change function switch is detected, the pre-activation warning could include a message such as “you are about to change the function.” Such a warning can provide the operator an opportunity to confirm the action about to be taken before the input device is contacted.

A control interface 10 according to the disclosure may use one sensor 18, an array of sensors, or more than one array of sensors to detect the attributes of objects in the sensing field(s). The processor 12 is connected to the sensors 18, array of sensors or more than one array of sensors and the stored values of valid attempts to actuate an input device 20, 22, 24 on the control interface 10 correspond to values generated by a human hand or foot detected by the sensor(s) or sensor array employed on the control device 10. It will be apparent that different sensor configurations will generate different data and the stored values must correspond to what is sensed by the sensor configuration connected to the processor 12. In a control interface 10 having more than one input device 20, 22, 24, one sensor 18 may be used to detect the attributes of objects 30 in the sensing field 28 and a library or look up table of criteria corresponding to valid actuation attempts may be constructed from values corresponding to the size, position and motion of objects in the sensing field as detected by the sensor configuration being used. The stored criteria and algorithm of the disclosed control interface 10 can be adjusted for a hand operated control interface 10 or for a foot operated control interface. The stored criteria and algorithm may also be adjusted for use with a foot switch supported on a floor or a control interface supported in a position to be actuated by a hand.

In a control interface 10 that employs an emitter such as an LED 16, the intensity and pattern of light emitted will vary depending upon the number of LEDs 16, their position on the control interface 10 and the power applied to the LED(s) 16 by the processor 12. The number, position and power of the LEDs 16 can be selected to produce a desired sensing field 28. The light generated by the LED(s) 16 and reflecting off objects 30 in the sensing field 28 will generate the data employed in the algorithm used to distinguish valid actuation attempts from other objects that may enter the sensing field 28, such as objects dropped on a footswitch. The stored values may include ranges of values for each attribute of an object in the sensing field. For example in a control interface 10 configured as a footswitch, stored values of object profile may include a range of values encompassing a reasonable range of profiles corresponding to different size human feet encased by shoes. Stored values of object position may include a range of positions relative to an input device 20, 22, 24 including a minimum distance from the input device 20, 22, 24. Stored values of object motion may include a maximum and minimum velocity or speed of an object 30 within the sensing field 28. The algorithm will include steps comparing data corresponding to one or more attributes of an object 30 in the sensing field 28 to one or more stored values and include the step of generating a control signal only when at least one, at least two, or all the attributes of an object 30 in the sensing field 28 are within a range of values corresponding to a valid attempt to actuate an input device 20, 22, 24.

The library of stored values corresponding to valid attempts to actuate an input device 20, 22, 24 can be assembled for each control interface 10 configuration and use environment. For example, a control interface 10 having the same physical configuration, sensor configuration and LED configuration may be provided with a different set of stored values and algorithm allowing the control interface 10 to be foot actuated or hand actuated. Control interfaces having different physical configurations, number of input devices, sensor configurations and/or LED configurations will necessarily require different sets of stored values and algorithms to reliably detect valid attempts to actuate an input device. According to aspects of the disclosure, the stored values and algorithm are designed to allow the control interface 10 to discriminate between valid attempts to actuate an input device and all other objects in the sensing field, while allowing the control interface 10 to function as expected.

FIGS. 3-6 illustrate a control interface configured as a footswitch. The control interface 10 includes a body or structure that rests on the floor and supports three input devices 20, 24, 20. Two of the input devices 20 are foot pedals connected to switches that detect the position of the pedals relative to the body of the control interface 10 and provide a variable output signal. A button type switch 24 is located on the body of the control interface 10 between the two pedals 20. A shoe 30 corresponding to a human foot is illustrated in proximity to one of the pedals 20. The sensing field(s) 28 of the control interface 10 include overlapping regions within which attributes of the shoe-clad foot can be detected. As shown in FIGS. 3-6, the range of positions 34, 36 of the foot relative to each of the three control devices 20, 24, 20 corresponding to a valid attempt to actuate each control device is distinct from the range of positions 34, 36 of the foot attempting to actuate the other of the control devices. Measured attributes of the foot in each position 34, 36 can be used to assemble the library of values corresponding to valid attempts to actuate each of the three control devices 20, 22, 24. The position values 34, 36 in particular can be used to distinguish an attempt to actuate one of the control devices 20, 22, 24 from an attempt to actuate the other control devices.

As shown in FIG. 8, the control interface algorithm may include a “lockout” function where the input device(s) 20, 22, 24 are not enabled until a valid attempt to actuate the corresponding input device is detected. Here “enabled” means that the output of the input device is active and actuation of the input device will generate an output signal and initiate a function of the connected equipment and “disabled” means the output of the input device is not active and actuation of the input device will not generate an output signal to initiate a function of the connected equipment. The lockout function of a control interface 10 can be used to disable the input device(s) when some detected combination of attributes is outside a set of stored values for any of the input devices. For example, if the input device is actuated (pedal depressed, switch position changed), but no valid attempt to actuate the input device has been detected, the input device remains disabled. This might occur if some object is resting on or against the control device but an operator is not present. The lockout function may be used to prevent inadvertent actuation of an input device by an object dropped on a footswitch for example.

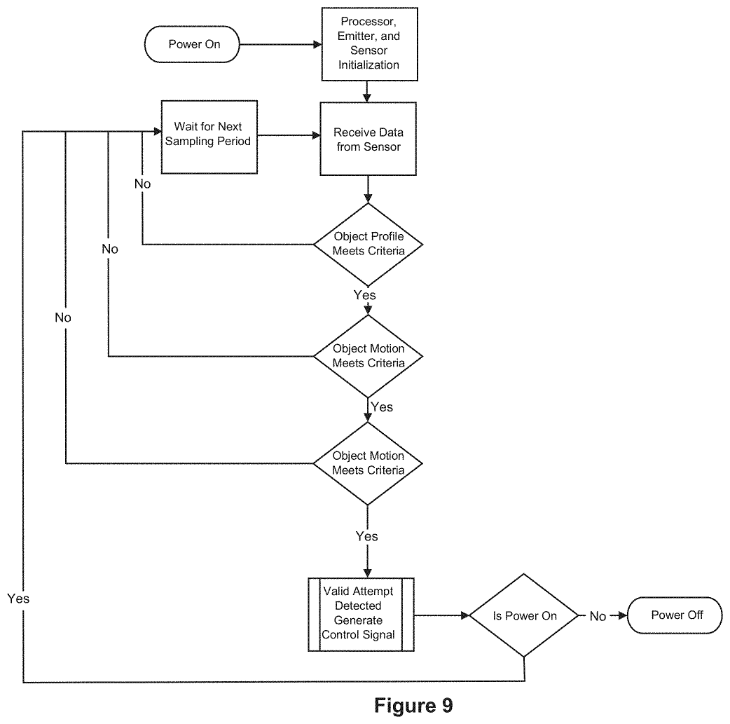

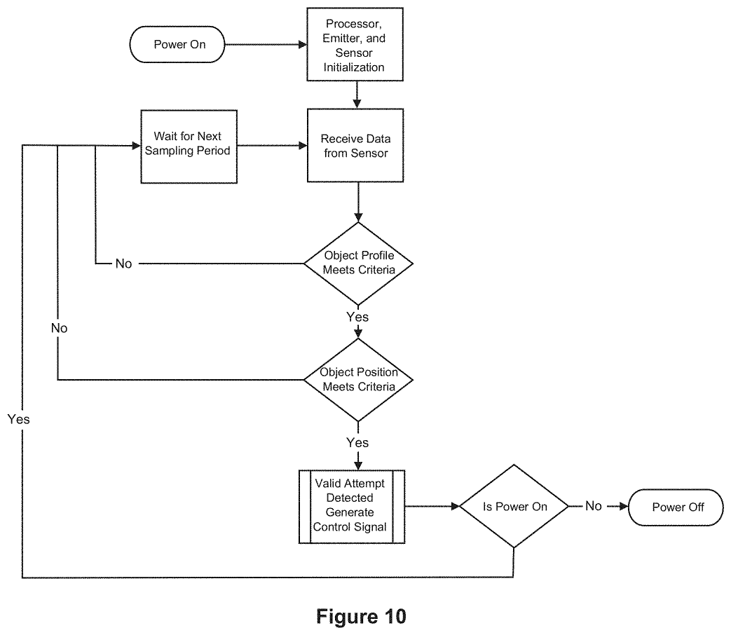

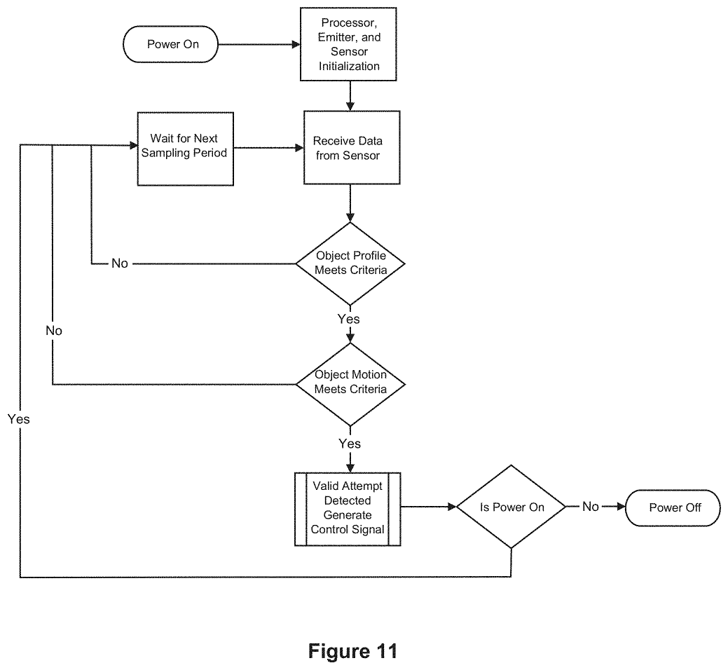

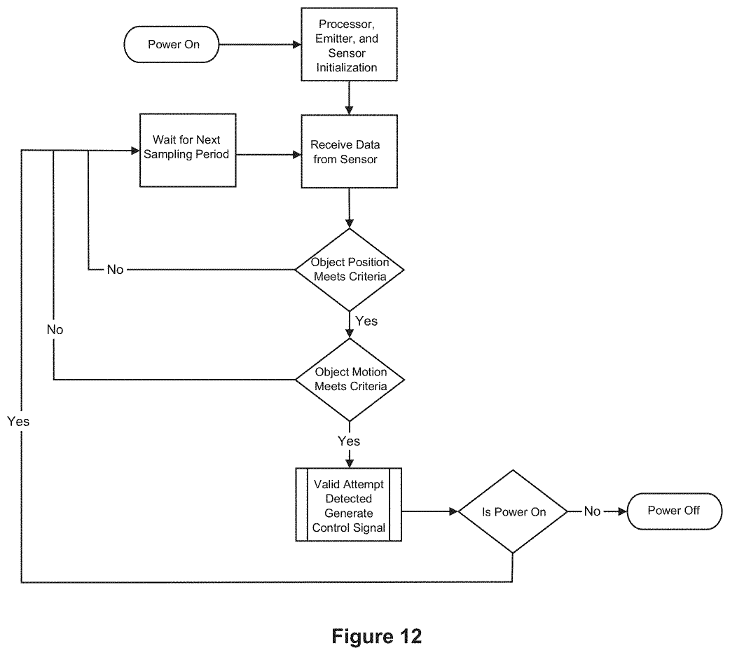

FIG. 9 is an alternative illustration of an algorithm for use in an embodiment of the disclosed control interface 10. In the algorithm of FIG. 9 the sensed attributes of profile, position, and motion are compared to stored values for each attribute that correspond to a valid attempt to actuate an input device. In the algorithm of FIG. 9 the control signal is only generated if all three attributes of the object match or are within the stored values. FIG. 10 illustrates a first variation of the algorithm of FIG. 9 in which only the profile and position attributes of the object are compared to stored values for profile and position. FIG. 11 illustrates a second variation of the algorithm of FIG. 9 in which the profile and motion attributes of the object are compared to stored values for profile and motion. FIG. 12 illustrates a third variation of the algorithm of FIG. 9 in which the position and motion attributes of the object are compared to stored values for position and motion. FIGS. 10-12 illustrate ways in which the algorithm(s) run in a control interface can be varied according to the intended use of the control interface.

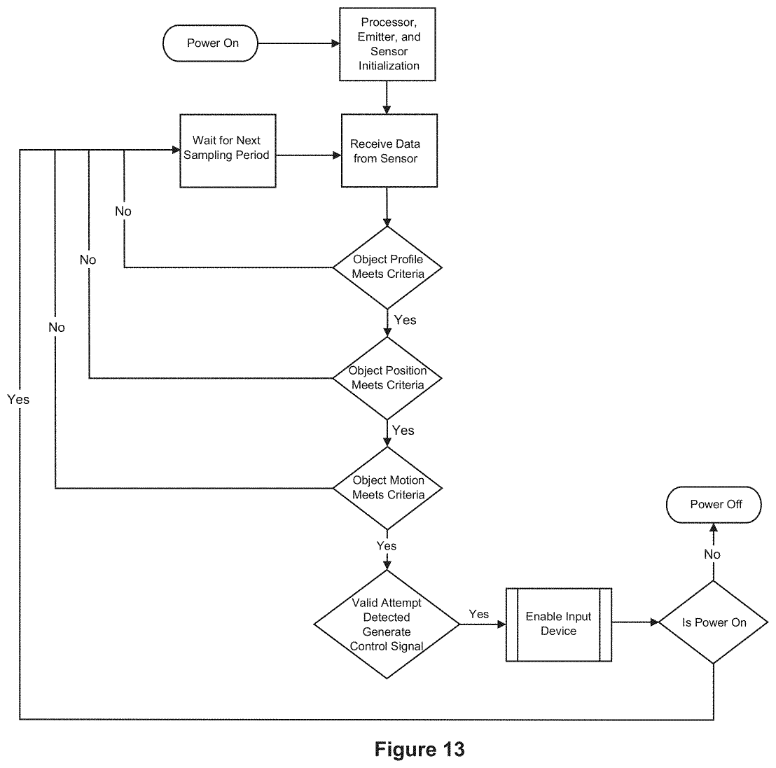

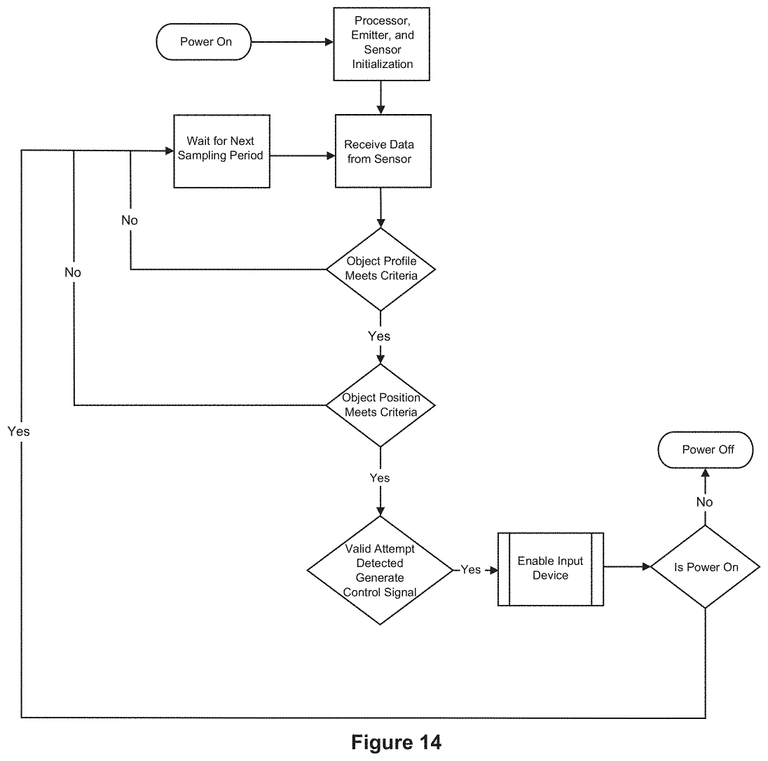

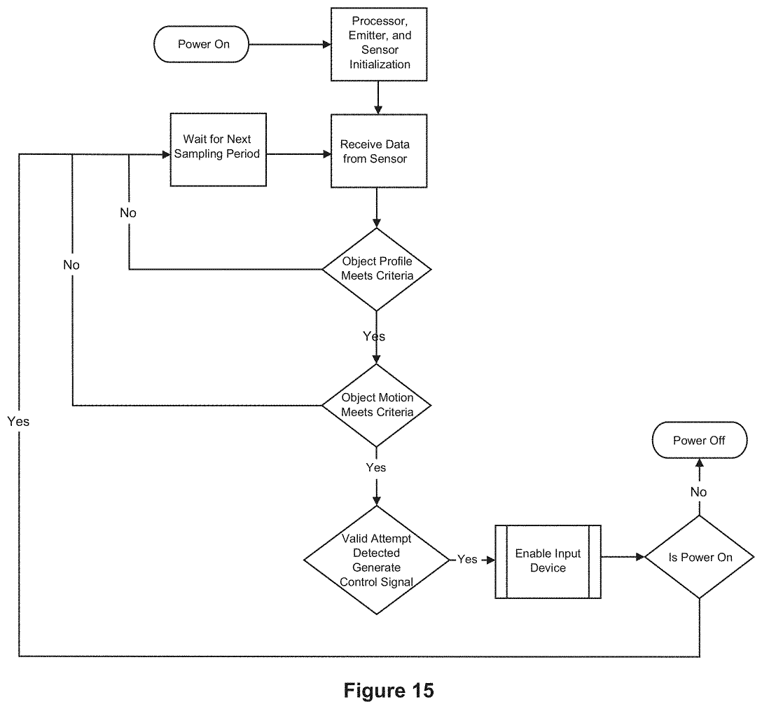

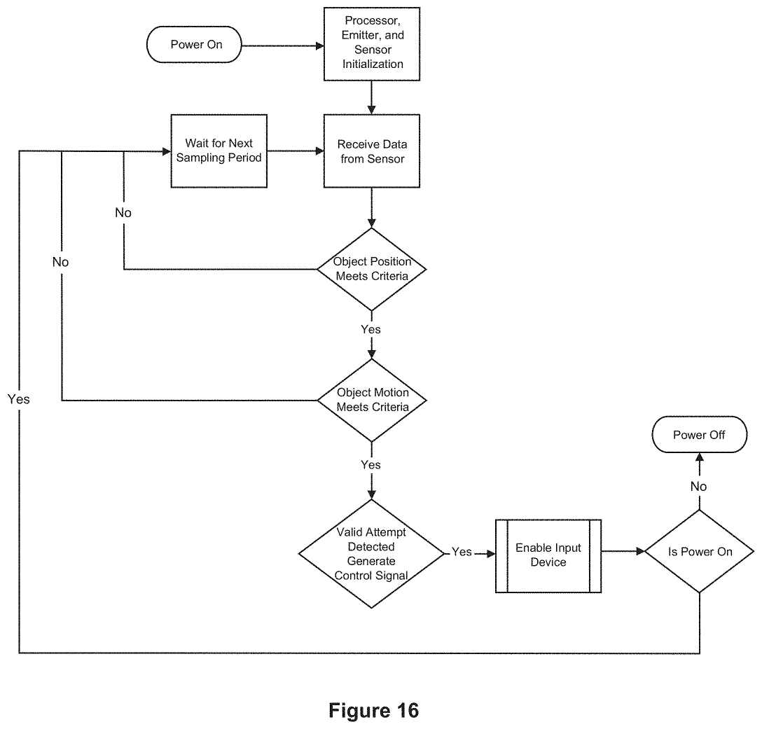

FIG. 13 is an alternative illustration of an algorithm for use in an embodiment of a control device 10. In the algorithm of FIG. 13 the sensed attributes of profile, position, and motion are compared to stored values for each attribute that correspond to a valid attempt to actuate an input device. In the algorithm of FIG. 13 the control signal is only generated and used to enable an input device if all three attributes of the object match or are within the stored values. FIG. 14 illustrates a first variation of the algorithm of FIG. 13 in which only the profile and position attributes of the object are compared to stored values for profile and position. FIG. 15 illustrates a second variation of the algorithm of FIG. 13 in which the profile and motion attributes of the object are compared to stored values for profile and motion. FIG. 16 illustrates a third variation of the algorithm of FIG. 13 in which the position and motion attributes of the object are compared to stored values for position and motion. FIGS. 14-16 illustrate ways in which the algorithm(s) run in a control interface can be varied according to the intended use of the control interface. The algorithms for each input device can be made more, or less restrictive according to the intended use and customer specification.

In variations of the algorithm such as those illustrated in FIGS. 10-12 and 14-16 that use two object attributes, the control interface may be configured to collect only the attributes used in the comparison and the stored values may include only the attributes used in the comparison. In a control interface with more than one input device, an algorithm such as those illustrated in FIGS. 7-16 would be used for each input device. The algorithm for each input device would run in series or parallel and when the attributes of an object in the sensing field(s) meet the criteria used in the algorithm for an input device, then the control signal is generated for the input device. The algorithms for each input device need not be identical. An algorithm such as that of FIG. 9 could be used for one input device and an algorithm such as that of FIG. 10 could be used for another input device. Thus a control interface according to the disclosure can have a wide variety of configurations that utilize one, two, or all three attributes of an object in the sensing field(s) to detect valid attempts to actuate each input device.

What is claimed:

1. A control interface comprising:
a base supporting at least one input device responsive to contact by a user to generate an output signal,
a sensor supported on the base, said sensor having a sensing field extending above the base and detecting the profile, position, or motion of objects in the sensing field,
a processor programmed to:
receive data corresponding to the profile, position or motion of an object in the sensing field;
compare said data representing the profile, position, or motion of the object to stored values of profile, position, or motion representing a valid operator attempt to actuate the input device; and
generate a control signal when the profile, position, or motion of the object meet the criteria for a valid operator attempt;
a communications link connected to the control interface and operable to communicate the control signal and output signal,
wherein said control signal is generated before any contact with the input device.

2. The control interface of claim 1, wherein the processor can enable or disable the input device, when the input device is disabled contact with the input device does not generate the output signal and when the input device is enabled contact with the input device generates the output signal, said processor is programmed to disable the input device until a valid operator attempt is detected and when a valid operator attempt is detected, to use the control signal to enable the input device.

3. The control interface of claim 1, wherein the stored values include:
a profile of an object corresponding to a valid operator attempt.

4. The control interface of claim 1, wherein the stored values include:
a range of positions of the object relative to the at least one input device corresponding to a valid operator attempt.

5. The control interface of claim 1, wherein the stored values include:
direction and speed of movement of the object within the sensing field corresponding to a valid operator attempt.

6. The control interface of claim 1, wherein the control signal is used to communicate to an operator that the valid operator attempt is detected.

7. The control interface of claim 1, wherein said sensor field extends in three dimensions from the sensor, and the position and motion of an object in the sensing field are detected in three dimensions.

8. The control interface of claim 1, wherein the at least one input device comprises a plurality of input devices and said stored values include a profile, position or motion of an object in the sensing field representative of a valid operator attempt for each of the plurality of input devices, wherein a valid operator attempt for each of the plurality of input devices cannot be a valid operator attempt for another of the plurality of input devices, and said processor is programmed to:
compare the data corresponding to the profile, position or motion of an object in the sensing field to the stored values for a valid operator attempt for each of the plurality of input devices; and
generate a control signal when the profile, position or motion of an object correspond to a valid operator attempt for one of the plurality of input devices,
wherein said control signal is used to communicate to an operator that a valid operator attempt is detected corresponding to one of the plurality of input devices, identifying the one of the plurality of input devices for which a valid operator attempt has been detected, and that the one of the plurality of input devices for which a valid operator attempt has been detected is enabled.

9. The control interface of claim 8, wherein the processor is programmed to:
validate an operator attempt for one input device at a time; and
prevent operation of the input devices other than the input device for which the valid operator attempt has been detected.

10. The control interface of claim 8, wherein the same sensing field is used to detect operator attempts for each of the plurality of input devices.

11. The control interface of claim 8, wherein a plurality of sensors are used to validate operator attempts for each of the plurality of input devices, each of said sensors having a sensing field, and the data corresponding to the profile, position or motion of an object is collected in each of the sensing fields, said stored values for a valid operator attempt for each of said plurality of input devices include profile, position or motion information for the object in each of the sensing fields.

12. The control interface of claim 1, wherein the processor is programmed to:
receive data corresponding to the profile, position and motion of an object in the sensing field;
compare said data representing the profile, position, and motion of the object to stored values of profile, position, or motion representing a valid operator attempt to actuate the input device; and
generate a control signal when the profile, position, and motion of the object meet are within the stored values for a valid operator attempt.

13. The control interface of claim 1, wherein the processor is programmed to:
receive data corresponding to two of the profile, position and motion of an object in the sensing field;
compare said data representing two of the profile, position, and motion of the object to stored values of two of the profile, position, or motion representing a valid operator attempt to actuate the input device; and
generate a control signal when two of the profile, position, and motion of the object meet the criteria for a valid operator attempt.
